(19) United States
(12) Patent Application Publication
(10) Pub. No.: US 2017/0119253 A1
(43) Pub. Date: May 4, 2017

(54) APPARATUS AND PROCESSING METHOD FOR ACQUIRING OBJECT INFORMATION

(71) Applicant: CANON KABUSHIKI KAISHA, Tokyo (JP)

(72) Inventors: Nobuhito Suehira, Tokyo (JP); Takuro Miyasato, Tokyo (JP)

(21) Appl. No.: 15/333,399

(22) Filed: Oct. 25, 2016

(30) Foreign Application Priority Data
Nov. 2, 2015 (JP) 2015-215939

(51) Int. Cl. A61B 5/00; A61B 8/15

(52) U.S. Cl. CPC A61B 5/0097; A61B 5/489; A61B 8/15

(57) ABSTRACT

An apparatus for acquiring object information according to the present invention includes an information acquisition unit acquiring characteristic information inside the object, wherein the information acquisition unit generates intensity distribution data corresponding to an initial sound pressure distribution, acquires, from values included in the intensity distribution data, a plurality of representative values corresponding to propagation distances of the light, and acquires an effective attenuation coefficient inside the object on the basis of the plurality of representative values.

### BACKGROUND OF THE INVENTION

Field of the Invention

The present invention relates to an apparatus and a processing method for acquiring object information.

Description of the Related Art

In recent years, photoacoustic tomography has been proposed as one of optical imaging techniques. An apparatus employing the photoacoustic tomography radiates pulsed light on a test object and propagates and diffuses the pulsed light in the test object. The apparatus detects an acoustic wave generated from an absorber having absorbed light energy (a photoacoustic wave), and performs signal processing. In this manner the apparatus can acquire, and then image, characteristic information related to optical characteristic values inside the test object.

In order to calculate an absorption coefficient distribution, which is an optical characteristic inside the test object, from the photoacoustic wave, it is necessary to calculate a distribution of an amount of light radiated on the absorber. However, since light introduced into the test object is absorbed and diffused, it is difficult to estimate the amount of light radiated on the absorber. Therefore, there are cases where, in the photoacoustic tomography, a light energy absorption density distribution, which is obtained by multiplying the absorption coefficient distribution by the light amount, is imaged.

Japanese Patent Application Laid-open No. 2009-018153 discloses a method in which an effective attenuation coefficient of a medium is calculated through measuring a sound pressure each time a light radiation position is changed in relation to a single absorber, and the effective attenuation coefficient is used to obtain a light amount distribution of a test object; then, by dividing a light energy absorption density distribution by the light amount distribution, an absorption coefficient distribution is obtained.

Patent Literature 1: Japanese Patent Application Laid-open No. 2009-018153

### SUMMARY OF THE INVENTION

In the photoacoustic tomography, values used in the calculation of a light amount distribution, e.g. an effective attenuation coefficient, are necessary for the acquisition of characteristic information, and there is a demand for a method which enables more satisfactory acquisition of these values.

The present invention has been devised in view of these problems. An object of the present invention is to satisfactorily acquire values necessary for calculating a light amount distribution inside an object in the photoacoustic tomography.

The present invention provides an apparatus for acquiring object information, the apparatus comprising:

an information acquisition unit configured to acquire characteristic information relating to an inside of the object using an electric signal output from an element, the element receiving an acoustic wave generated when an object is irradiated with light and outputting the electric signal, wherein

the information acquisition unit is configured to
generate intensity distribution data corresponding to an initial sound pressure distribution inside the object,
acquire, from values included in the intensity distribution data, a plurality of representative values corresponding to propagation distances of the light inside the object, and
acquire an effective attenuation coefficient inside the object on the basis of the plurality of representative values.

The present invention also provides a processing method for acquiring characteristic information relating to an inside of an object using an electric signal acquired by receiving an acoustic wave generated when light is radiated on the object, the processing method comprising:

generating intensity distribution data corresponding to an initial sound pressure distribution inside the object;

acquiring, from values included in the intensity distribution data, a plurality of representative values corresponding to propagation distances of the light inside the object; and

acquiring an effective attenuation coefficient inside the object on the basis of the plurality of representative values.

According to the present invention, it is possible to satisfactorily acquire values necessary for calculating a light amount distribution inside an object in the photoacoustic tomography.

Further features of the present invention will become apparent from the following description of exemplary embodiments with reference to the attached drawings.

### BRIEF DESCRIPTION OF THE DRAWINGS

FIGS. 1A to 1C are diagrams for explaining a principle in a first embodiment;

FIGS. 2A and 2B are diagrams of a photoacoustic apparatus in the first embodiment;

FIG. 3 is a flowchart for explaining a light attenuation calculating method in the first embodiment;

FIGS. 4A and 4B are diagrams for explaining projected image creation in the first embodiment; and

FIGS. 5A and 5B are diagrams of a photoacoustic apparatus in a second embodiment.

### DESCRIPTION OF THE EMBODIMENTS

Preferred embodiments of the present invention are explained below with reference to the drawings. However, the dimensions, the materials, the shapes, a relative arrangement, and the like of components described blew should be changed as appropriate according to the configuration and various conditions of an apparatus applied with the invention and are not meant to limit the scope of the present invention to the description explained below.

The present invention relates to a technique for detecting an acoustic wave propagating from an object and generating and acquiring characteristic information relating to an inside of the object. Therefore, the present invention is grasped as an object information acquiring apparatus or a control method therefor or an object information acquiring method or a signal processing method. The present invention is also grasped as a computer program for causing an information processing apparatus including hardware resources such as a CPU and a memory to execute the methods and a storage medium having the computer program stored therein.

The object information acquiring apparatus of the present invention includes an apparatus that radiates light (an electromagnetic wave) on an object to thereby receive an acoustic wave generated in the object and acquires characteristic information of the object as image data and makes use of a photoacoustic effect. In this case, the characteristic information is information concerning characteristic values generated using a reception signal obtained by receiving a photoacoustic wave and respectively corresponding to a plurality of positions in the object. Note that, in the present invention, an effective attenuation characteristic is also included in the characteristic information.

The characteristic information acquired by photoacoustic measurement is a value reflecting an absorption rate of light energy. The characteristic information includes, for example, a generation source of an acoustic wave generated by light radiation, an initial sound pressure in the object, or light energy absorption density and an absorption coefficient derived from the initial sound pressure, or the concentration of a substance configuring a tissue. An oxygen saturation distribution can be calculated by calculating oxygenated hemoglobin concentration and reduced hemoglobin concentration as substance concentration. Glucose concentration, collagen concentration, melanin concentration, volume fractions of fat and water, and the like can also be calculated. A two-dimensional or three-dimensional characteristic information distribution is obtained on the basis of characteristic information in the positions in the object. Distribution data can be generated as image data. The characteristic information may be calculated as distribution information in the positions in the object rather than as numerical value data. That is, distribution information such as an initial sound pressure distribution, an energy absorption density distribution, an absorption coefficient distribution, and an oxygen saturation distribution may be used as the object information. These kinds of information can be collectively referred to as optical characteristic information distribution.

The acoustic wave in the present invention is typically an ultrasound wave and includes an elastic wave called sound wave or acoustic wave. An electric signal converted from the acoustic wave by a probe or the like is referred to as acoustic signal. However, the description of ultrasound wave or acoustic wave in this specification does not intend to limit wavelength of the elastic waves. The acoustic wave generated by the photoacoustic effect is called photoacoustic wave or photo-ultrasound wave. An electric signal deriving from the photoacoustic wave is referred to as photoacoustic signal.

<Examination>

As explained above, in Japanese Patent Application Laid-open No. 2009-018153, the photoacoustic measurement is performed in the plurality of light radiation positions to calculate the effective attenuation coefficient. However, changing the light radiation positions and performing measurement a plurality of times leads to complication of processing and an increase in a measurement time. There is also a calculation method for detecting light transmitted and diffused after being radiated on the object. However, in this case, an apparatus configuration for the light detection is necessary. There is also a method of using statistical data according to an age of a subject and a measured region. However, in this case, the effective attenuation coefficient is likely to be different from an actual attenuation characteristic of the subject.

Therefore, there is a desire to acquire an effective attenuation characteristic of the subject using a photoacoustic image obtained in a photoacoustic measurement situation. A method of acquiring an effective attenuation coefficient from a photoacoustic image of a subject including a plurality of absorbers is explained below. In acquiring the effective attenuation coefficient, factors due to the absorbers such as types and sizes of the absorbers and factors due to the apparatus such as characteristics of the probe, disposition of the probe, frequency band of the probe and a circuit, and the like are related.

### First Embodiment

(Principle)

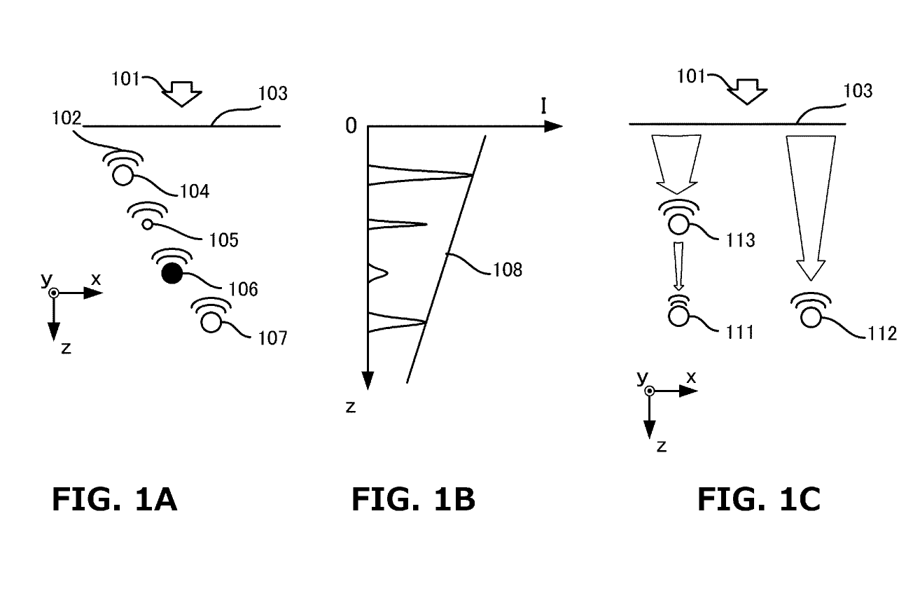

First, a principle of the present invention is explained. FIG. 1A is a schematic photoacoustic tomograpahic image obtained when a plurality of absorbers 104 to 107 are present at depths different from one another from a surface 103 on the inside of a test object (an object). Light is uniformly radiated from the surface 103 toward a z direction, which is a depth direction. An effective attenuation coefficient of a medium is fixed in a region shown in the figure. Acoustic wave 102 generated from the absorbers is received by a probe (not shown in the figure) disposed on the surface.

When the test object is an organism, the surface is the body surface, for example. As the absorbers, blood vessels (arteries and veins) can be assumed. In FIGS. 1A and 1B, a plurality of blood vessels are present in a region to be imaged. A difference in an absorption coefficient is caused by a difference between oxygen saturations of the arteries and the veins. In the case of FIG. 1A, the absorber 104 and the absorber 107 have the same absorption coefficient and the same thickness. The absorber 105 has an absorption coefficient same as the absorption coefficient of the absorber 104 but is smaller than the absorber 104. When the structure of an absorber is smaller or larger than a target size like the absorber 105, since the absorber deviates from a band of the probe, the intensity of a photoacoustic signal decreases. Compared with the absorber 104, the absorber 106 has the same degree of size but has a small absorption coefficient.

A sound pressure (P) of an acoustic wave obtained in the photoacoustic tomography is represented by Formula (1).

P=Γ·μₐ·Φ (1)

Γ represents a Gruneisen coefficient, which is an elastic characteristic value and is obtained by dividing a product of a coefficient of cubical expansion (β) and a square of sonic speed (c) by specific heat (Cp). μₐ represents an absorption coefficient of an absorber. Φ represents a light amount in a local region (a light amount radiated on the absorber).

The light amount is represented by Formula (2) using a function z of depth.

[Math. 1]

φ=φ₀EXP(−μ_{eff}z) (2)

Φ₀ is incident light on the surface. Formula (2) indicates that the incident light is exponentially attenuated as the incident light travels in the depth direction. μ_{eff} represents an average effective attenuation coefficient in the medium. Note that, as explained below, depth z may be considered as representing a propagation distance of light.

Next, a method of calculating the average effective attenuation coefficient of the medium from a photoacoustic image in FIG. 1A is explained. FIG. 1B is a graph obtained by one-dimensionally projecting a tomographic image of photoacoustic measurement shown in FIG. 1A. The vertical axis indicates the depth (z) in the object and corresponds to the z axis of FIG. 1A. The depth z indicates a propagation distance of light inside the object. The horizontal axis indicates signal intensity (I). The horizontal axis (I) is logarithmical representation.

An attenuation characteristic calculation function 108 for calculating an effective attenuation coefficient is created on the basis of a curve having a plurality of peaks and showing a one-dimensional projected image. The attenuation characteristic calculation function can be considered a function for approximating a light attenuation characteristic corresponding to the effective attenuation coefficient. In FIG. 1B, as the attenuation characteristic calculation function, a linear function is created in which at least two peaks of the plurality of peaks are connected and the other peaks do not cross. However, a method of calculating the attenuation characteristic calculation function is not limited to this. It is sufficient to calculate a function based on a signal intensity value from the absorber and calculate the effective attenuation coefficient from Formula (2). When the attenuation characteristic calculation function 108 is created from a two-dimensional tomographic image having intensity of each of pixels, a pixel having maximum intensity is selected out of pixels at the same depth.

The sizes and the absorption coefficients of the absorbers in FIG. 1A, the peaks of the curve in FIG. 1B, and the attenuation characteristic calculation function 108 are compared and examined. When the absorber 104 near the surface and the absorber 107 in the depth are compared, since the light amount is large near the surface, the signal intensity of the absorber 104 is larger. Since the absorber 105 has the small structure, a frequency generated by the absorber 105 is high. As a result, the sensitivity of the probe decreases and the signal intensity decreases. Since the absorber 106 has a small absorption coefficient, the signal intensity is smaller than the attenuation characteristic calculation function.

In this way, when the plurality of absorbers are present in a certain range, the attenuation characteristic calculation function can be acquired from the one-dimensional projected image by the same type of the absorbers that can be detected at desired sensitivity. Therefore, it is desirable to select radiated light with which a target absorber emits a strong signal. A tilt of the attenuation characteristic calculation function obtained in this way is μ_{eff} from Formula (2) because the horizontal axis is logarithmical representation. In this way, the average effective attenuation coefficient of the medium can be calculated.

However, the calculation method is not limited to this calculation method as long as the effective attenuation coefficient can be calculated. For example, the signal intensity does not have to be the logarithmical representation and may be fit by an exponential function. In the above example, the maximum of the signal intensity is selected. However, a representative value other than the maximum may be acquired. For example, the representative value may be calculated by excluding a clearly abnormal value. For example, artifact during reconstruction, a multiple reflection component on an object surface or an interface between the object and a holding member, or the like can be mixed as an abnormal value. In some case, a median or an average may be adopted as the representative value. Concerning a pixel group (a position group) at the same propagation distance from a light radiation position, a histogram based on a signal intensity may be created to calculate the representative value.

Note that the creation of the one-dimensional projected image has effects described below. First, as shown in FIG. 1C, it is assumed that a first absorber 111 and a second absorber 112 having same degrees of conditions such as sizes and absorption coefficients are present at the same depth. A third absorber 113 is present between the surface 103 and the first absorber 111. An absorber is absent between the surface 103 and the second absorber 112. In this case, a light amount radiated on the second absorber 112 is a value obtained by attenuating a light amount radiated on the object surface according to the effective attenuation coefficient. On the other hand, a light amount radiated on the first absorber 111 is not only reduced according to the effective attenuation coefficient but also reduced by light absorption by the third absorber 113. As a result, an acoustic wave generated from the first absorber 111 is smaller than an acoustic wave generated from the second absorber 112. Therefore, when the one-dimensional projected image is created, a signal from the second absorber 112 is selected.

That is, according to this method, an absorber having more desirable conditions can be selected from a plurality of absorbers present at the same depth in two-dimensional (or three-dimensional) data. The obtained attenuation characteristic calculation function is a line connecting signals from the same type of the absorbers present at different depths. Conversely, when an attenuation coefficient is calculated focusing on one target, a substance having an unexpected characteristic is sometimes present in a route. In this way, calculation accuracy of the effective attenuation coefficient is improved by performing one-dimensional projection.

(Photoacoustic Apparatus)

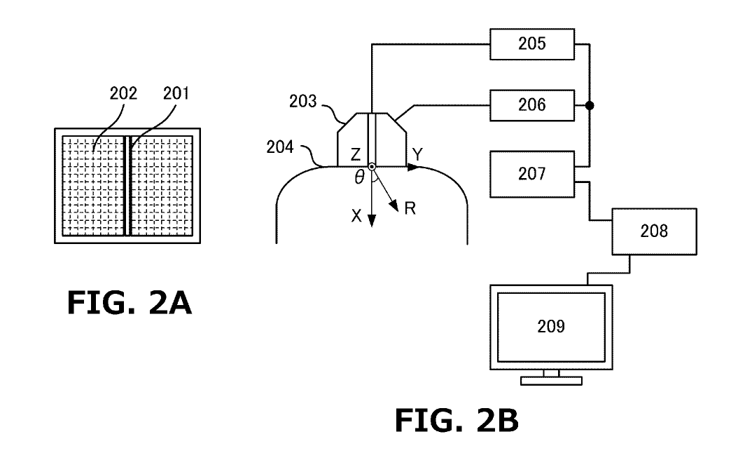

As an example of a photoacoustic apparatus of the present invention, an apparatus including a probe of a handheld type is explained. FIG. 2A is a diagram showing disposition of a probe and a light radiating section in the handheld probe. A linear light radiating section 201 is present in the center. Two-dimensional probes 202 are disposed on both sides of the light radiating section 201.

FIG. 2B is a diagram of the configuration of the photoacoustic apparatus. The apparatus includes a photoacoustic probe 203, a light control section 205, an ultrasound control section 206, an apparatus control section 207, an information acquirer 208 as an acquisition unit, and a display section 209. The photoacoustic probe 203 is disposed such that a probe surface is in contact with a test object 204. The photoacoustic apparatus is capable of performing photoacoustic measurement by synchronizing reception timing of the probe 202 with light radiated from the light radiating section 201. It is possible to perform ultrasound measurement if the probe 202 performs transmission and reception of ultrasound. Note that separate probes may be prepared from photoacoustic measurement and for ultrasound echo measurement.

(Light Radiating Section)

The light radiating section 201 is a linear portion that radiates pulsed light radiated on the test object 204. The pulsed light is led from a light source to the light radiating section 201 by a bundle fiber. That is, a plurality of point light sources are linearly arranged to thereby form a linear light source. Note that the structure of the light radiating section 201 is not limited to this. Light may be enlarged by a lens or the like and formed as a linear light source by a slit. A radiation shape is formed as the linear shape in order to form a two-dimensional tomographic image. However, a configuration for radiating light in a wide region of the object may be adopted.

As the light source, a laser beam source is desirable in order to obtain a large output. However, the light source may be a light emitting diode, a flash lamp, or the like. When the laser is used, various lasers such as a solid-state laser, a gas laser, a dye laser, and a semiconductor laser can be used. Timing, a waveform, intensity, and the like of radiation of light are controlled by the light control section 205.

In order to effectively generate the photoacoustic wave, light has to be radiated in a sufficiently short time according to a thermal characteristic of the test object. When the test object is an organism, pulse width of the pulsed light generated from the light source is suitably approximately 10 to 50 nanoseconds. Wavelength of the pulsed light is desirably wavelength for propagating the light to the inside of the test object. Specifically, in the case of the organism, the wavelength is 700 nm or more and 1100 nm or less. A titanium sapphire laser, which is a solid-state laser, is used. The wavelength is set to 760 nm or 800 nm. If lights having a plurality of wavelengths can be radiated, it is possible to calculate substance concentration making use of a difference in a degree of absorption at each of the wavelengths.

(Probe)

The two-dimensional probe 202 is an element that performs reception of a photoacoustic wave and transmission and reception of ultrasound and is also called transducer. Examples of the element include PZT (as piezoelectric ceramics) and CMUT (as capacitive micro machine probe). The handheld-type probe 202 in this embodiment is configured by, for example, 64×10 elements on one side. The element receives an acoustic wave and outputs an electric signal.

A signal converted into an electric signal by the probe is transferred to the ultrasound control section 206, amplified by an amplifier, converted into a digital signal by an A/D converter, and sent to the apparatus control section 207. Note that the reception timing of the acoustic wave is controlled by the apparatus control section 207 to synchronize with light radiation. A band of the probe 202 is, for example, 2 MHz to 5 HMz. 2048 samplings are performed at a sampling frequency of 50 MHz. Data is signed 12-bit data. When an ultrasound image is generated, time gain control or the like for compensating for attenuation according to depth may be performed.

(Information Acquirer)

The information acquirer 208 generates a photoacoustic image of the inside of the object through image reconstruction using a photoacoustic signal deriving from a photoacoustic wave. When acquiring an ultrasound attenuation characteristic through ultrasound measurement, the information acquirer 208 processes an ultrasound signal deriving from an ultrasound echo. The information acquirer 208 further carries out desired processing such as signal correction. The information acquirer 208 can be configured by an information processing device including a processor and a memory. Functions of the information acquirer 208 can be realized by modules of a computer program operating in the processor. The information acquirer 208 may be configured by an information processing device common to the light control section and the ultrasound control section.

(Signal Processing)

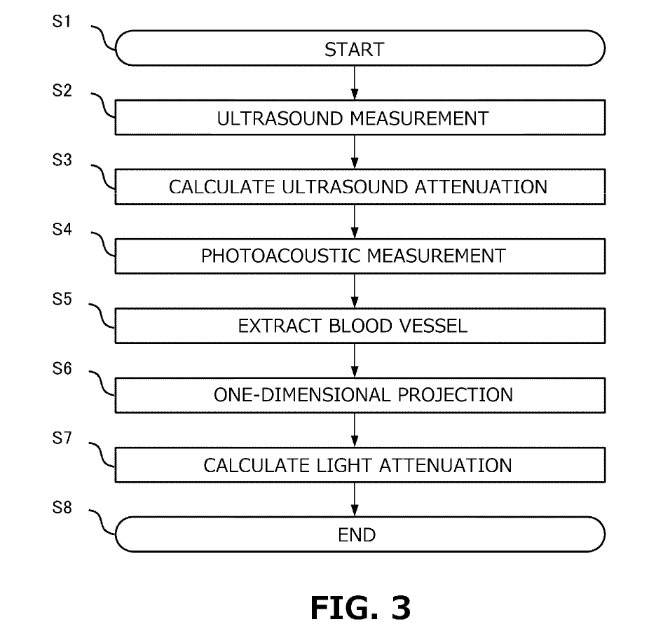

Signal processing is explained with reference to a flowchart of FIG. 3.

In step S1, measurement is started. In this state, a surgeon holds the photoacoustic probe 203 and brings the probe 202 into contact with a test object via gel for acoustic matching.

In step S2, ultrasound measurement is performed. Note that this processing is performed to acquire an attenuation characteristic inside the test object. However, a general value may be used according to characteristics of the test object (e.g., age, sex, and a region if the test object is an organism) or a value obtained by prior measurement may be used. Such a value can be acquired by being stored in a memory (not shown in the figure) in advance or being input from a user interface. In that case, steps S2 and S3 are unnecessary.

In this step, the probe 202 transmits ultrasound and receives a reflected signal from the test object. At this point, it is desirable to appropriately set a focus position or the like and perform beam forming. A necessary frequency may be set according to a region to be measured. The information acquirer 208 generates, as an ultrasound image, a B-Scan image in a direction parallel to the linear light radiating section 201. Since the probe 202 is two-dimensionally disposed, a three-dimensional ultrasound image is obtained. Advanced correction such as the time gain control is not performed. However, when the photoacoustic apparatus also functions as an ultrasound imaging device, an ultrasound image is presented to a user in addition to the photoacoustic image. In such a case, the time gain control may be separately performed.

In step S3, the information acquirer 208 calculates an ultrasound attenuation characteristic inside the object. The attenuation characteristic is used for correction of an attenuation amount of sound pressure generated in the absorber that is attenuated until the sound pressure reaches the probe 202. The attenuation of the ultrasound is represented like Formula (3).

[Math. 2]

A=A₀EXP(−αfz) (3)

Coefficients are α: an attenuation coefficient, A₀: initial sound pressure, f: a transmission frequency, and Z: a propagation distance.

The information acquirer 208 extracts a uniform area of a scatterer from the ultrasound image obtained in step S2, acquires an attenuation degree of luminance with respect to a depth direction of the area, and calculates an attenuation characteristic using the attenuation degree. Note that when the probe is not used, a general value of a region of the test object may be used. For example, the value is 0.5 dB/cmHMz. Since the ultrasound attenuation depends on a frequency, the ultrasound attenuation may be calculated from ultrasound images acquired at a plurality of frequencies. The photoacoustic wave is often a frequency component lower than ultrasound for ultrasound measurement. Therefore, the ultrasound signal obtained by the ultrasound measurement may be corrected to be attenuated in a desired frequency band.

In step S4, photoacoustic measurement is performed. The light radiating section 201 radiates pulsed light. The probe 202 receives a photoacoustic wave in synchronization with the radiation of the pulsed light. By changing the wavelength of radiated light and performing the photoacoustic measurement according to necessity, it is possible to selectively image an artery, a vein, a tumor, and the like. Note that S/N is improved by performing the photoacoustic measurement a plurality of times and adding up signals. This photoacoustic image reflects an initial sound pressure distribution inside the object at the time when the light is radiated. As the photoacoustic image, the initial sound pressure distribution may be used or an energy absorption density distribution specified by initial sound pressure and an absorption coefficient may be used. The initial sound pressure distribution or the energy absorption density distribution is a set of values of signal intensity in each of positions. Therefore, the initial sound pressure distribution or the energy absorption density distribution can also be called intensity distribution data.

The information acquirer 208 applies a reconstruction method such as a universal back projection method or a phasing addition method to the photoacoustic signal to generate a photoacoustic image. In this case, it is desirable to compensate for ultrasound attenuation according to a target position and a distance to the transducer using the attenuation characteristic acquired in step S3. The generation of the photoacoustic image in this step may be performed at accuracy in the same degree as generation of a photoacoustic image finally presented to the user. However, the generation of the photoacoustic image may be performed by processing simpler than generation of a final image. The simpler processing is, for example, processing for curtailing data and accelerating calculation. In the first place, the photoacoustic measurement in step S4 may be simplified by reducing measurement positions. In that case, in order to acquire a photoacoustic wave for generating the final image, light radiation different from the light radiation in step S4 is necessary.

In step S5, the information acquirer 208 extracts a blood vessel from the photoacoustic image. In this step, even if there are absorbers having different shapes such as a tumor and a blood vessel, it is possible to extract an absorber having a desired shape. A blood vessel having thickness in a fixed range is extracted as a desired absorber.

For the extraction of a blood vessel, a general method can be used. For example, there is a method of determining and binarizing a threshold and determining, as blood vessels, places where signals are present. Further, only a blood vessel having desired thickness is extracted using a band-pass filer or the like. A range of the thickness is, for example, 0.5 mm to 3 mm. When detection sensitivity of an absorber is different depending on a position of an image, the detection sensitivity may be multiplied by a correction coefficient. Consequently, it is possible to acquire a three-dimensional photoacoustic image in which an absorber (a blood vessel) having a desired shape is extracted. However, since the absorber present in the object is reflected on the photoacoustic image, even when this step is not performed, it is possible to calculate the effective attenuation coefficient at a certain degree of accuracy using signal intensity in each of positions.

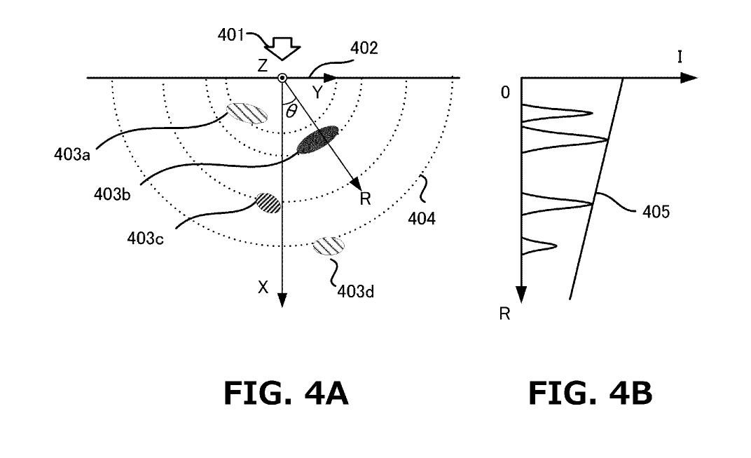

In step S6, the information acquirer 208 creates a one-dimensional projected image from the blood vessel image. This step is explained with reference to FIGS. 4A and 4B. FIG. 4A is a maximum intensity projection image (MIP image) generated on the basis of a three-dimensional photoacoustic image. In generating the MIP image, first, the information acquirer 208 sets, as a Z axis (a direction perpendicular to the paper surface), a direction in which the linear light radiating section 201 extends. The information acquirer 208 generates two-dimensional tomographic images in a plurality of positions in the Z direction. The information acquirer 208 compares signal intensities of pixels present in the same positions among the plurality of two-dimensional tomographic images and acquires maximum intensity. The MIP Image is obtained by performing this processing in all positions of the two-dimensional tomographic images. Note that, in generating an image through the maximum intensity projection, correction such as exclusion of abnormal values may be performed.

Subsequently, the information acquirer 208 sets, in the three-dimensional photoacoustic image, a columnar coordinate system in which the direction in which the linear light radiating section 201 extends is the Z axis (the direction perpendicular to the paper surface), a distance from the light source is R, and an angle from an axis (an X axis) in the depth direction is θ. In the MIP image, the coordinate system is represented by a polar coordinate in which a distance R from the origin is a moving radius and an angle is θ.

The information acquirer 208 selects a pixel having the maximum signal intensity I for each of pixel groups having the same degree of the distance R and plot the signal intensity with a logarithm to obtain a graph shown in FIG. 4B. For example, in FIG. 4A, positions having the distance R equal to the distance R to an absorber 403d are indicated by a dotted line 404. The maximum intensity on the dotted line 404 is equivalent to the bottom peak in FIG. 4B.

In the example shown in FIGS. 4A and 4B, since the linear light source is used, the distance R is used in the plot. This is the same meaning as a plot in which the depth z is used when light is radiated in a wide range. In other words, a pixel having a largest value is selected for each of pixels having equal or the same degree of a light propagation distance inside the object. When the light is linearly radiated on the object as shown in FIGS. 4A and 4B the light source is indicated in a dot shape in a projected image, a light propagation distance from an incident position is represented by R. This is the same in the case of a point light source. On the other hand, a propagation distance is represented by the depth z when light is radiated in a wide range in a plane shape. Therefore, if z is replaced with R, Formula (2) can be applied to FIGS. 4A and 4B.

In step S7, the information acquirer 208 calculates an effective attenuation of light. That is, in the logarithmic plot shown in FIG. 4B, the information acquirer 208 draws an attenuation characteristic calculation function 405 by a linear function not crossing other signals and calculates a tilt of the attenuation characteristic calculation function 405. When the signal intensity is not plotted with the logarithm, an attenuation characteristic calculation function can also be drawn by an exponential function or the like. Note that, when the signal intensity can be fit by a strict analytic solution, a polynomial or the like may be used. In this way, an average effective attenuation coefficient μ_{eff} of the medium can be acquired. Note that, when the attenuation characteristic calculation function is drawn, a depth range may be limited in order to avoid the influence of a strong signal generated near the surface.

The effective attenuation coefficient obtained in this way can be used for calculation of a light amount distribution. The light amount distribution can be used in calculating an absorption coefficient distribution from an initial sound pressure distribution. The initial sound pressure used at this point may be the same as the initial sound pressure acquired in step S4 or may be acquired by performing photoacoustic measurement again. Alternatively, the acquired effective attenuation function may be used for correction of an already generated photoacoustic image. The acquired effective attenuation coefficient may be stored in the memory in association with information concerning the subject.

As explained above, according to the present invention, the effective attenuation coefficient inside the object can be calculated by the arithmetic operation using the photoacoustic image. Therefore, since it is unnecessary to radiate lights from a plurality of places and acquire information, easy and quick processing is possible. When the photoacoustic signal and the photoacoustic image used to acquire the effective attenuation coefficient are used for imaging of the inside of the object, processing can be made efficient.

### Second Embodiment

(Photoacoustic Apparatus)

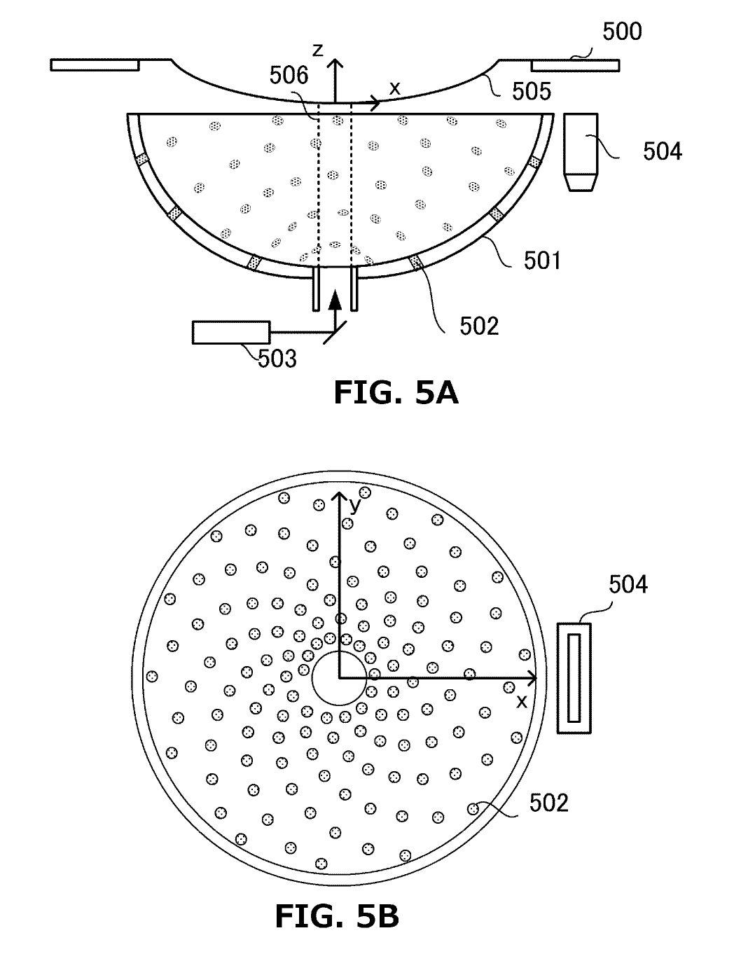

A photoacoustic apparatus for breast measurement in this embodiment is shown in FIGS. 5A and 5B. FIG. 5A is a sectional view of a holding member for a test object and a measuring device for an acoustic wave in the photoacoustic apparatus. FIG. 5B is a plan view of a probe seen through the holding member from an upper surface.

Concerning the measuring device, five hundred and twelve probes 502 are spirally arranged along the inner surface of a semispherical container 501. A space through which measurement light from a light radiating section 503 is transmitted is provided in the bottom of the semispherical container 501. The measurement light is radiated on a test object from a negative direction of a z axis. The test object is disposed in a holding member 505. As the holding member 505, a material that transmits light and an acoustic wave like polyethylene terephthalate is desirable. An acoustic matching material (e.g., water or castor oil) is filled in the inside of the semispherical container 501 and the inside of the holding member 505 according to necessity.

A relative positional relation between the semispherical container 501 and the test object is changed by an XY stage (not shown in the figures). In positions where the semispherical container 501 is scanned by the XY stage, substantially parallel pulsed light 506 is radiated. The probes 502 detect a photoacoustic wave. The information acquirer 208 reconfigures data obtained by the probes 502, whereby a three-dimensional photoacoustic image is obtained. Note that ultrasound echo measurement used in acquiring an acoustic characteristic inside the object is performed by a linear ultrasound probe 504. The linear ultrasound probe 504 is capable of scanning.

(Light Radiating Section)

In order to effectively generate photosound, it is necessary to radiate light in a sufficiently short time according to a thermal characteristic of the test object. When the test object is an organism, pulse width of pulsed light generated from a light source is suitably approximately 10 to 50 nanoseconds. A titanium sapphire laser, which is a solid-state laser, is used. Two wavelengths of 760 nm and 800 nm are used in order to measure oxygen saturation.

(Probes for Photosound)

The probes 502 perform reception of a photoacoustic wave. A CMUT (as capacitive micro machine probe) is used. As a single element, the probe has an opening of φ3 mm and a band of the probe is 0.5 to 5 MHz. Since a low frequency is included in the band, it is possible to acquire a satisfactory image even in a blood vessel having thickness of approximately 3 mm. That is, a situation in which the blood vessel is seen in a ring shape less easily occurs. 2048 samplings are performed at a sampling frequency of 50 MHz. Data is signed 12-bit data.

(Linear Ultrasound Probe)

The linear ultrasound probe 504 can perform transmission and reception of ultrasound and obtain a shape image. As such an element, PZT (as piezoelectric ceramics) is used. The number of elements is 256. A band of the element is 5 to 10 MHz. 2048 samplings are performed at a sampling frequency of 50 MHz. Data is signed 12-bit data.

(Coordinate System)

In this embodiment, a coordinate system is an orthogonal coordinate system in which an optical axis direction of radiated light is a z axis and a crossing point with the holding member 505 is an origin. However, a method of setting a coordinate is not limited to this. The coordinate may be a spherical surface coordinate in which a crossing point of the radiated light and a hemispherical surface is an origin. When the spherical surface coordinate system is used, calculation is easy, for example, when it can be regarded that a point light source is present at the origin.

(Processing Flow)

Differences from the first embodiment are particularly explained with reference to the flowchart of FIG. 3. At the measurement start in step S1, a breast is disposed in the holding member 505. In the ultrasound measurement in step S2, the linear ultrasound probe 504 is scanned in an x direction. As a result, B-scan images parallel to a zy plane are obtained. By combining the obtained images, a three-dimensional ultrasound image is obtained. In ultrasound attenuation characteristic calculation in step S3, distances from the holding member 505 to a target in xy coordinate positions are used. This is because ultrasound is not attenuated much in an acoustic matching material and is attenuated in a process of propagation in a medium.

In the photoacoustic measurement in step S4, the light radiating section 201 radiates pulsed light while spirally moving the XY stage. Since the radiating section 201 performs the measurement while moving the XY stage, a reconfigured three-dimensional photoacoustic image is equivalent to an image obtained when substantially parallel light radiation is performed. Note that a photoacoustic image may be obtained in every radiation of the pulsed light.

In the creation of the one-dimensional projected image from the blood vessel image in step S7, it should be noted that attenuation of light starts from the position of the holding member 505. That is, the position of the holding member 505 is set as a zero point in xy positions. In the creation of the two-dimensional projected image (the MIP image), maximum signal intensity of a photoacoustic image may be projected on an xz plane. Subsequently, the maximum signal intensity is projected on the z axis from the MIP image. Note that a range where a curvature of the holding member 505 is large may be excluded. The calculation of the light attenuation characteristic in step S8 may be individually performed by dividing a region of the object into a plurality of regions on an xy plane. By adopting the configuration explained above, it is possible to calculate an effective attenuation coefficient of light from the photoacoustic image.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-215939, filed on Nov. 2, 2015, which is hereby incorporated by reference herein in its entirety.

What is claimed is:

1. An apparatus for acquiring object information, the apparatus comprising:
an information acquisition unit configured to acquire characteristic information relating to an inside of the object using an electric signal output from an element, the element receiving an acoustic wave generated when an object is irradiated with light and outputting the electric signal, wherein
the information acquisition unit is configured to
generate intensity distribution data corresponding to an initial sound pressure distribution inside the object,
acquire, from values included in the intensity distribution data, a plurality of representative values corresponding to propagation distances of the light inside the object, and
acquire an effective attenuation coefficient inside the object on the basis of the plurality of representative values.

2. The apparatus according to claim 1, wherein the information acquisition unit is configured to use, as the intensity distribution data, the initial sound pressure distribution or an energy absorption density distribution inside the object.

3. The apparatus according to claim 2, wherein the information acquisition unit is configured to generate the intensity distribution data in two dimensions and acquire the representative value from the two-dimensional intensity distribution data.

4. The apparatus according to claim 2, wherein the information acquisition unit is configured to generate the intensity distribution data in three dimensions, generate the intensity distribution data in two dimensions through maximum projection using the three-dimensional intensity distribution data, and acquire the representative value from the two-dimensional intensity distribution data.

5. The apparatus according to claim 1, wherein the information acquisition unit is configured to compare the values included in the intensity distribution data and select a maximum in each of the propagation distances of the light inside the object to thereby acquire the plurality of representative values.

6. The apparatus according to claim 5, wherein the light is radiated on the object in a plane shape, and the propagation distance of the light is represented as depth from a surface of the object.

7. The apparatus according to claim 5, wherein the intensity distribution data is two-dimensional intensity distribution data, a position where the light is made incident on the object is indicated in a dot shape in the two-dimensional intensity distribution data, and the propagation distance of the light is represented as a distance from the position where the light is made incident.

8. The apparatus according to claim 1, wherein the information acquisition unit is configured to create an attenuation characteristic calculation function representing an attenuation characteristic inside the object on the basis of the plurality of representative values to acquire the effective attenuation coefficient.

9. The apparatus according to claim 8, wherein the information acquisition unit is configured to create the attenuation characteristic calculation function on the basis of a relation between the plurality of representative values and the propagation distances.

10. The apparatus according to claim 9, wherein the information acquisition unit is configured to create the attenuation characteristic calculation function on the basis of a function indicating that a light amount is exponentially attenuated when the light propagates inside the object.

11. The apparatus according to claim 1, wherein
the object includes a plurality of absorbers, and
the information acquisition unit is configured to acquire the representative value using values deriving from the plurality of absorbers in the intensity distribution data.

12. The apparatus according to claim 1, wherein the information acquisition unit is configured to
acquire a light amount distribution in the object using a light amount of the light radiated on the object and the effective attenuation coefficient and
acquire an optical characteristic information distribution inside the object using the light amount distribution and the electric signal.

13. The apparatus according to claim 12, wherein the information acquisition unit is configured to acquire the optical characteristic information distribution using the electric signal used to generate the intensity distribution data.

14. The apparatus according to claim 12, wherein the information acquisition unit is configured to acquire the optical characteristic information distribution using an electric signal different from the electric signal used to generate the intensity distribution data.

15. The apparatus according to claim 1, further comprising a memory configured to store the effective attenuation coefficient.

16. The apparatus according to claim 1, wherein the information acquisition unit is configured to generate the intensity distribution data using an ultrasound attenuation characteristic obtained by transmitting and receiving ultrasound to and from the inside of the object.

17. The apparatus according to claim 16, wherein the element is also used for the transmission and reception of the ultrasound for acquiring the ultrasound attenuation characteristic.

18. The apparatus according to claim 16, further comprising an ultrasound probe configured to transmit and receive the ultrasound to and from the inside of the object.

19. A processing method for acquiring characteristic information relating to an inside of an object using an electric signal acquired by receiving an acoustic wave generated when light is radiated on the object, the processing method comprising:
generating intensity distribution data corresponding to an initial sound pressure distribution inside the object;
acquiring, from values included in the intensity distribution data, a plurality of representative values corresponding to propagation distances of the light inside the object; and
acquiring an effective attenuation coefficient inside the object on the basis of the plurality of representative values.

20. A non-transitory storage medium having stored therein a computer program for causing a computer to execute the processing method according to claim 19.
